(19) United States
(12) Patent Application Publication
(10) Pub. No.: US 2006/0121098 A1
(43) Pub. Date: Jun. 8, 2006

(54) Dermal defense system and method of use

(76) Inventors: Randall P. Mendoza, San Antonio, TX (US); Lance A. FitzSimon, San Antonio, TX (US); Eduardo M. Medellin, Dallas, TX (US)

Correspondence Address: KAMMER BROWNING PLLC, 7700 BROADWAY, SUITE 202, SAN ANTONIO, TX, 78209, US

(21) Appl. No.: 11/274,051

(22) Filed: Nov. 15, 2005

Related U.S. Application Data
(60) Provisional application No. 60/628,014, filed on Nov. 15, 2004.

(51) Int. Cl. A61K 9/70; B65H 1/00

(52) U.S. Cl. 424/443; 221/33

(57) ABSTRACT

A system that provides an antimicrobial, dermal defense for hand-mediated functions. The system includes microbicidal towelettes and dispensers for towelette delivery near dermal microbial sources. More specifically, the system includes towelettes impregnated with a specific antimicrobial composition and a dispenser unit containing the towelettes positioned next to a microbe-containing source; and may be used in conjunction with a refuse container for disposal of the towelettes. The user grasps the towelette from the dispenser unit, positions the towelette as a barrier between the user's hand and the microbe-contaminated source, grasps the microbe-contaminated source with the towelette, and properly disposes of the towelette after use.

### CROSS REFERENCE TO RELATED APPLICATIONS

This application claims the benefit under Title 35 United States Code § 119(e) of U.S. Provisional Application No. 60/628,014 filed Nov. 15, 2004, the full disclosure of which is incorporated herein by reference.

### BACKGROUND OF THE INVENTION

1. Field of the Invention

The present invention relates generally to systems that provide antimicrobial dermal defense for hands. The present invention relates more specifically to systems for use in association with microbicidal towelettes and dispensers for towelette delivery near dermal microbial sources. The present invention also relates to methods of use for such systems.

2. Description of the Related Art

Vast numbers of microbial organisms impact human health. Infection by viruses and bacteria causes a wide variety of illnesses. Media focus on epidemics of West Nile virus, AIDs, influenza, food poisoning; staphylococcal and streptococcal infections, and a seemingly endless list of other microbial illnesses is increasing public awareness of the dangers of microbial infections and the need to control the spread of microbes. Particularly for those illnesses which are transmitted by skin contact between one human being and another, the relationship between dermal defense and public health is critical.

Much effort is therefore directed to environmental cleanliness to reduce or inhibit the spread of microbes. It is well known that the washing of hard surfaces, food (e.g., fruit or vegetables) and skin (especially the hands) with antimicrobial or non-medicated soap, can remove many viruses and bacteria from the washed surfaces. This microbial removal is due to the surfactancy of the soap and the mechanical friction of the washing procedure. Therefore, it is recommended that people wash frequently to reduce the spread of viruses and bacteria. In healthcare facilities, hand washing has been shown to be the single most effective means of preventing the spread of nosocomial infections.

Bacteria found on the skin can be divided into two groups: resident and transient bacteria. Resident bacteria are established as permanent micro-colonies on the surface and outermost layers of the skin and play an important, helpful role in preventing the colonization of other, more harmful bacteria and fungi.

Transient bacteria are bacteria which are not part of the normal resident flora of the skin, but can be deposited when airborne contaminated material lands on the skin or when contaminated material is brought into physical contact with the skin. Both transient and resident bacteria are typically divided into two subclasses: Gram positive and Gram negative. Gram positive bacteria include pathogens such as Staphylococcus aureus, Streptococcus pyogenes and Clostridium botulinum. Gram negative bacteria include pathogens such as Salmonella, Escherichia coli, Klebsiella, Haemophilus, Pseudomonas aeruginosa, Proteus and Shigella dysenteriae. Gram negative bacteria are generally distinguished from Gram positive by an additional protective cell membrane which generally results in the Gram negative bacteria being less susceptible to topical antibacterial soaps.

Antimicrobial cleansing products have been marketed in a variety of forms including deodorant soaps, hard surface cleansers, and surgical disinfectants. These traditional “rinse-off” antimicrobial products have been formulated to provide bacteria removal during washing. The antimicrobial soaps have also been shown to provide a residual effectiveness against Gram positive bacteria, but limited residual effectiveness versus Gram negative bacteria. By residual effectiveness it is meant that bacteria growth on a surface is controlled for some period of time following the washing/rinsing process. Antimicrobial liquid cleansers are disclosed in U.S. Pat. No. 4,847,072, Bissett et al., issued Jul. 11, 1989, U.S. Pat. No. 4,939,284, Degenhardt, issued Jul. 3, 1990 and U.S. Pat. No. 4,820,698, Degenhardt, issued Apr. 11, 1989 (all cited U.S. patents are incorporated herein by reference).

Many of these traditional antimicrobial products, especially the hard surface cleansers and surgical disinfectants, utilize high levels of alcohol and/or harsh surfactants which have been shown to dry out and irritate skin tissues. Ideal personal cleansers should gently cleanse the skin with little or no irritation, not leaving the skin overly dry after frequent use and preferably moisturizing the skin. Finally, these traditional antimicrobial compositions have generally been developed for use in a washing process that requires warm water. This limits their use to locations with readily available warm water.

Cleansing wipes have been used, in the past, to wash hands and face while traveling or in public or anytime water is not available. In fact, consumers have used absorbent sheets impregnated with topical compositions for this purpose. The problem inherent with the use of such wipes is the fact that subsequent to their use, any contact with a microbial source recontaminates the surface of the skin. Although the current market includes several antibacterial wipes, these wipes are intended and manufactured for the purpose of cleaning the hands as a substitute to hand washing and are not intended to serve as a dermal defense against recontamination and the subsequent spread of germs. Moreover, these wipes often contain harsh compounds that irritate the skin.

Applicants have found that antimicrobial towelettes which provide mildness and residual effectiveness versus Gram negative bacteria can be formulated by using known porous or absorbent sheets which are impregnated with an improved antimicrobial composition. This improved antimicrobial composition contains antimicrobial actives in combination with skin soothing compounds. Such a combination in a towelette provides a microbial barrier which does not irritate the skin.

It is now known that many forms of infectious disease, such as the common cold, are contracted and spread to others primarily by the hands. Conventional medical wisdom teaches that the best way to prevent contraction and transmission of most common bacterial and viral infections is by thoroughly and frequently cleansing the hands throughout the day, and primarily before touching the face or preparing food. In typical medical settings, the health care provider frequently uses latex gloves. This greater expense and inconvenience is warranted where the risk of infection is so great. However, in other settings where the risk of infection is not so great, this expense and inconvenience is prohibitive. There are many situations in daily life that call for an intermediate solution for the prevention of microbial infection of the hands. The average person routinely encounters situations where he or she is called upon to handle, grasp, or hold objects that have recently been handled by others in the public.

Cleansing and sanitizing an individual's hands using soap are generally effective for removing microbes that have accumulated on the hands throughout the day. For additional protection, an antibacterial soap may be used. Hand washing is sometimes followed by applying a topical sterilant such as antibacterial hand lotion to further protect the hands. Although these measures will help to control the spread of many pathogens residing on the skin, they are ineffective if not performed routinely and methodically. Unfortunately, routine use of antibacterial soaps and lotions may cause chapping of the skin or other undesirable reactions, especially for those persons who are sensitive to the active ingredients found in these products.

One of the most common environments in which microbes are transmitted is in public restroom facilities. In a public restroom, although soap and/or sterilant dispensers are provided, it is often the case that the supply is depleted. This is usually only discovered after the individual has attempted to activate the dispenser by manual means, which is likely contaminated by prior users. Without an antibacterial soap, mere rinsing of the hands with water is not effective for eliminating microbes on the skin.

Moreover, many individuals choose not to wash their hands. Upon exiting the restroom, germs from these individual's hands are frequently deposited on the door handle and are transmitted to the next person to exit. Thus, even with diligence, there is a substantial likelihood that an individual will leave a public restroom area with far greater microbial contamination than he or she had upon entering the restroom.

In a variety of different fields and businesses, there is a need for assuring that persons who enter certain areas have sanitized their hands prior to entry. Obvious examples include food preparers and health care workers, although there are other potential examples too numerous to list. The food service industry is a clear example of the need for employees to sanitize their hands prior to handling food, especially after their use of a restroom. Bacteria (e.g., E. coli) in restrooms, are easily transmitted among restaurant employees and unknowing customers without proper cleaning of the hands.

There is a need for a simple and inexpensive method to assure that persons who have washed their hands are not reinfected upon exiting the unsanitary area. Especially desirable is a system that is simple and inexpensive enough to allow it to be retrofitted into existing restrooms. Given the severe health impact of dermally-transmitted microbes such as Salmonella, Escherichia coli and Shigella, it would be highly desirable to have an antimicrobial dermal defense system which provides a skin transmission barrier to prevent re-contamination of cleansed skin surfaces by unsanitary elements of the environment. Additionally, such a system should be simple and economical to use, mild to the skin, and utilize a single-use, disposable product. Existing products have been unable to deliver a system which provides all of these benefits.

### SUMMARY OF THE INVENTION

The present invention provides a system for protecting the user's skin from surface microbial contamination in hand-mediated functions. The system includes a dispenser containing single-use antimicrobial towelettes located in a position conveniently accessible to the user prior to grasping a microbial-contaminated object or surface. The user grasps and gently pulls the towelette from the dispenser and uses it to provide a barrier between the hand and the contaminated object or surface, such as a public restroom door knob or handle.

The antimicrobial compounds contained in the towelette provide a transmission barrier for those microbes that are on the surface touched by the user. The antimicrobial towelettes of the present invention are mild to the skin, providing the user a protective barrier without additional available water. The antimicrobial towelette includes a sheet of absorbent material impregnated with an antimicrobial composition affording the user protection from transient microbe-laden surfaces. The towelette is sized and structured to be easily handled and to adequately cover the surface of the user's hand.

### BRIEF DESCRIPTION OF THE DRAWINGS

A more complete understanding of the objects and processes of the present invention may be had by reference to the following detailed description taken in conjunction with the accompanying drawings, wherein:

FIG. 1 shows an elevational view of a towelette dispenser which is suitable for use within the system and method of the present invention.

FIG. 2 shows an elevational view of the dispenser shown in FIG. 1 with the front panel lifted upward in the open position.

FIG. 3 shows a cross-sectional view (along A-A′ of FIG. 1) of the interior of the towelette dispenser.

FIG. 4 is a diagram of a preferred embodiment implementation of the system of the present invention in conjunction with a public restroom facility.

FIG. 5 is a plan view of the preferred embodiment implementation shown in FIG. 4 further showing the process for utilizing the system.

FIG. 6 is a plan view of a second preferred embodiment implementation of the present invention within a public dining facility.

FIG. 7 is a plan view of a third preferred embodiment implementation of the present invention in conjunction with a public telephone.

FIG. 8 is a plan view of a fourth preferred embodiment implementation of the present invention in conjunction with slot machines.

### DETAILED DESCRIPTION OF THE PREFERRED EMBODIMENTS

As summarized above, the present invention is directed to a system for use in association with microbicidal towelettes and dispensers for towelette delivery near dermal microbial sources. The present invention also relates to methods of use for such a system.

The present invention includes the following essential components: a basic dermal defense system, methods for utilizing the system, and applications of the basic system to specific environments. The basic dermal defense system includes antimicrobial towelettes, a towelette dispenser, an optimized location for the dispenser, the object to be handled, and a towelette disposal location. Methods for utilizing the system include: providing the dermal defense barrier (towelette), dispensing the barrier at a convenient location, using the barrier to handle an object, and disposing of the barrier. Exemplary applications of the basic system to specific environments include, but are not limited to: public restroom facilities, public dining facilities, public telephones, and public casino slot machines.

The Basic Dermal Defense Barrier

The antimicrobial composition is impregnated onto one or both sides of an absorbent sheet which may be formed from any woven or non-woven fiber or fiber mixture of sufficient wet strength and absorbency to hold an effective amount of the antimicrobial composition depending on the absorbent capacity of the sheet, such that the towel is moist to the touch but does not stay wet. It is preferred, from the standpoint of antimicrobial effectiveness and mildness, to employ a sheet with a high absorbent capacity (e.g., from about 5 to about 20 grams/gram, and preferably from about 9 to about 20 grams/gram). Additionally, it is important that the sheet possess sufficient tensile strength to reduce the likelihood of tearing the towelette, with subsequent loss of the microbial barrier.

In particular, woven or non-woven fabrics derived from carded fibrous webs composed of textile-length fibers with the majority of the fibers oriented predominantly in one direction are particularly suitable. The manufacture of materials suitable for retention of the antimicrobial compounds is well known in the art. The sheets are sized and shaped to cover the hand of an average user, approximately 4 inches wide by 8 inches long.

The absorbent sheets used in the present invention are impregnated with an antimicrobial composition suitable for contact with human skin. The term “antimicrobial composition” as used herein means a composition that may be suitable for application to the skin for the purpose of controlling the growth and viability of microbes. The antimicrobial composition of the present invention is made of a combination of antimicrobial actives, Methylparaben, Propylparaben, Lanolin, and Aloe Barbadensis, selected to satisfy the efficacy and mildness requirements of the invention and manufactured according to techniques well known in the art.

Specifically, the antimicrobial composition of a preferred embodiment of the present invention is a mixture comprised of the following components in the indicated ranges dissolved in an appropriate amount of purified water: 0.05-0.5% (w/v) Benzalkonium Chloride, 0.05-0.5% (w/v) Hymine (Benzethonium Chloride), 0.10-5.0% (w/v) Methylparaben, 0.01-0.5% (w/v) Propylparaben, 0.10-5.0% (w/v) Lanolin PEG 60, and 0.10-5.0% (w/v) Aloe Barbadensis. Benzalkonium Chloride and Benzethonium Chloride are known to be bactericidal against many organisms including Gram positive, Gram negative, and Acid fast bacteria. Additionally, the antiviral properties of Benzalkonium Chloride are known in the art. Methylparaben and Propylparaben are antimicrobial preservatives which are highly effective over a wide pH range and have a broad spectrum of antimicrobial activity, although most effective against yeasts and molds. The addition of Lanolin PEG 60 and Aloe Barbadensis in the presence of these antimicrobial actives did not negatively impact the inhibitory effects of the active ingredients individually or in combination. The Lanolin provides a water soluble emollient and also decreases the evaporation of the other components of the composition, whereas Aloe provides a hypoallergenic skin-soothing component for additional mildness to the skin.

Any manufacturing method suitable for the application of aqueous or aqueous/alcoholic impregnates, including flood coating, spray coating, metered dosing, or other techniques known in the art, can be used to impregnate the absorbent sheets of the present invention with the antimicrobial composition. After coating, the sheets are folded into stacks according to any of a number of configurations known in the art, such as interleaved, interleaved Z-fold, multi-fold, or center pull with perforations. The folded sheets are packaged in a moisture and vapor impermeable package having a tear away seal located on the bottom whereby the package may be opened and the towelettes subsequently dispensed.

The Basic Dermal Defense System

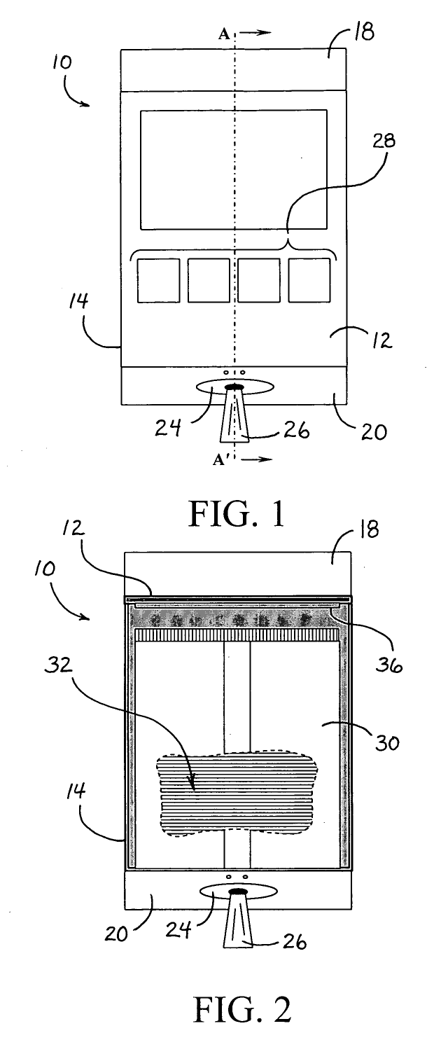

The basic dermal defense barrier is utilized within a system which provides for the convenient dispensing and disposal of the barrier. The primary component of such a system is a dispensing unit as shown in FIG. 1. Dispenser unit 10 is comprised of frame enclosure 14 and front panel 12. Frame enclosure 14 is a generally rectangular enclosure made up of top panel 18, bottom panel 20, and side panels (not shown here). The top panel 18 is angled to prevent the placement of foreign objects thereon. The front panel 12 of the dispenser unit 10 contains pictograms 28 which provide instructional information regarding usage of the dispenser unit. The bottom panel 20 of the frame enclosure 14 contains a dispenser port 24 through which a towelette 26 is dispensed.

An elevational view of the dispenser unit 10, with the front panel 12 in the open position is shown in FIG. 2. The front panel 12 is attached to the frame enclosure 14 by a hinge 36. Also shown in FIG. 2 is a cutaway view of the towelette package 30 through which can be seen the disposable folded towelettes 32. The front panel 12 is shown in a raised position pivoting on hinge 36, providing access to the towelette package 30.

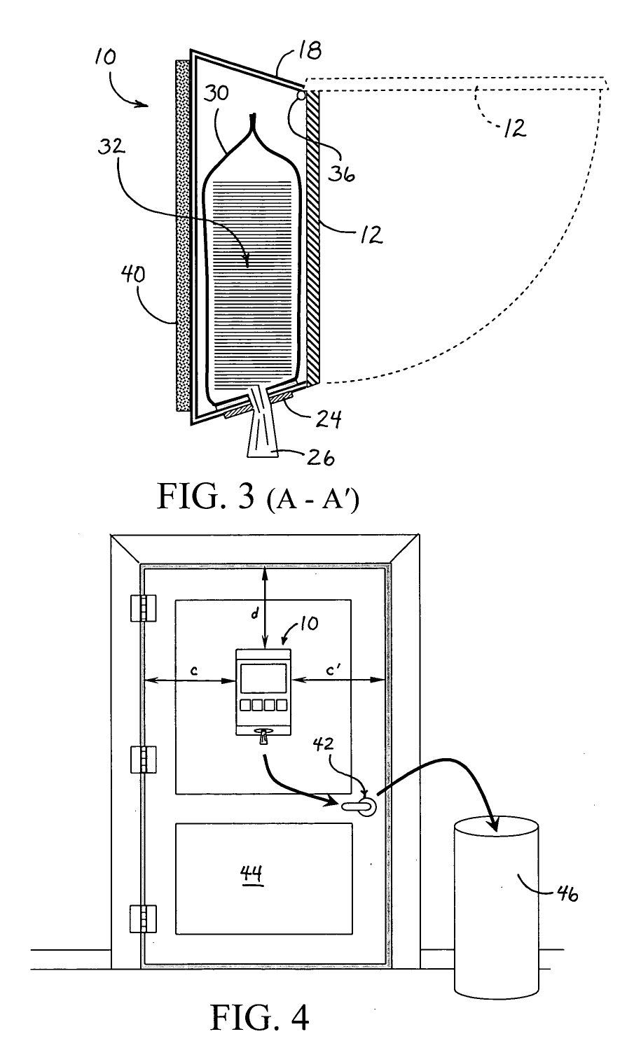

FIG. 3 is a cross-sectional view of the dispenser unit 10 along A-A′ of FIG. 1. In this view, attachment means 40 is shown positioned on the backside of dispenser unit 10. In this figure, the open position of the front panel 12 is shown by the dotted line.

Applications of the Basic System to Specific Environments

FIG. 4 is a diagram of a preferred embodiment of the system in a public restroom facility. The dispenser unit 10 is positioned on restroom door 44 at a preferred distance d of 22 inches from the top of a standard 80 inch door. The distances c and c′ vary according to the door width, with the object being to approximately center the dispenser unit 10 on the door 44. In this embodiment, the user pulls a towelette from the dispenser unit 10 and grasps the door handle 42 to open the door 44. After pulling the door open, the user disposes the towelette in a refuse container 46.

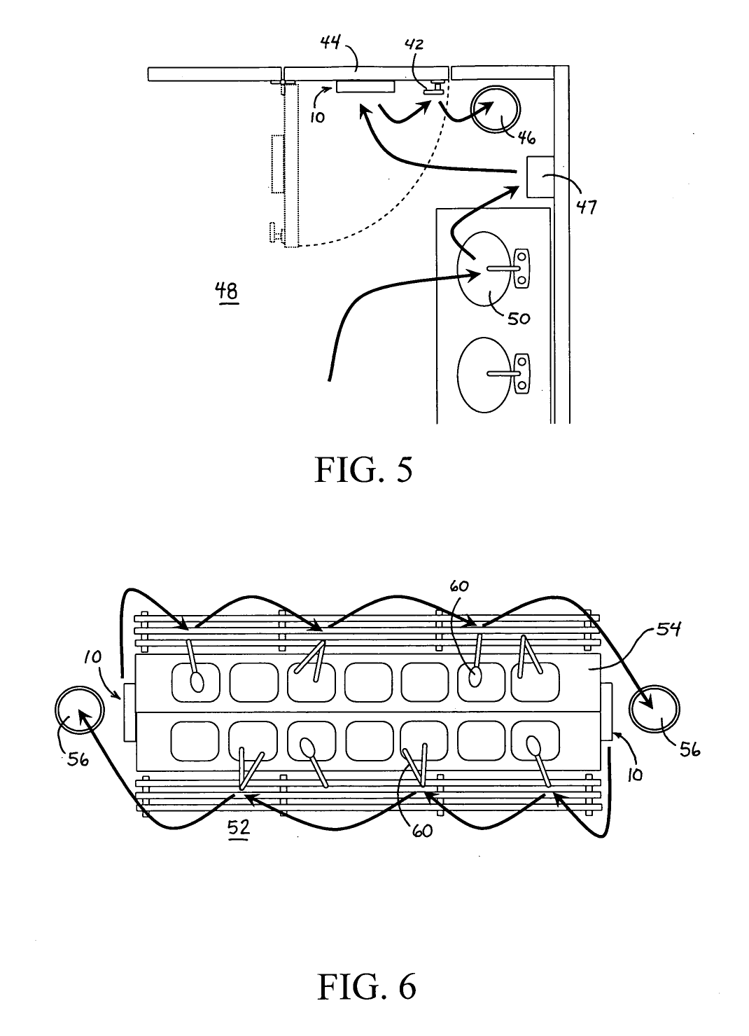

A typical configuration for use of the system in a public restroom facility 48 is shown in FIG. 5. After washing the hands at the wash sink 50 and drying the hands at the drying station 47, the user proceeds to the dispenser unit 10 positioned in conjunction with an exit door to the restroom facility 48. As described above, the user pulls a towelette from the dispenser unit and grasps the exit door handle 42 to open the door 44. After pulling the door open, the user disposes the towelette 26 in a refuse container 46.

FIG. 6 shows the use of the system in a second preferred embodiment in a public dining facility 52. Dispenser units 10 are positioned at starting points on the self-service food bar 54 such as a salad bar or a breakfast bar. The user pulls a towelette from a dispenser unit and uses it to grasp the service utensils 60 as needed along the food bar. The user proceeds through the food bar 54 and disposes of the towelette in a refuse container 56 positioned at an endpoint of the food bar 54.

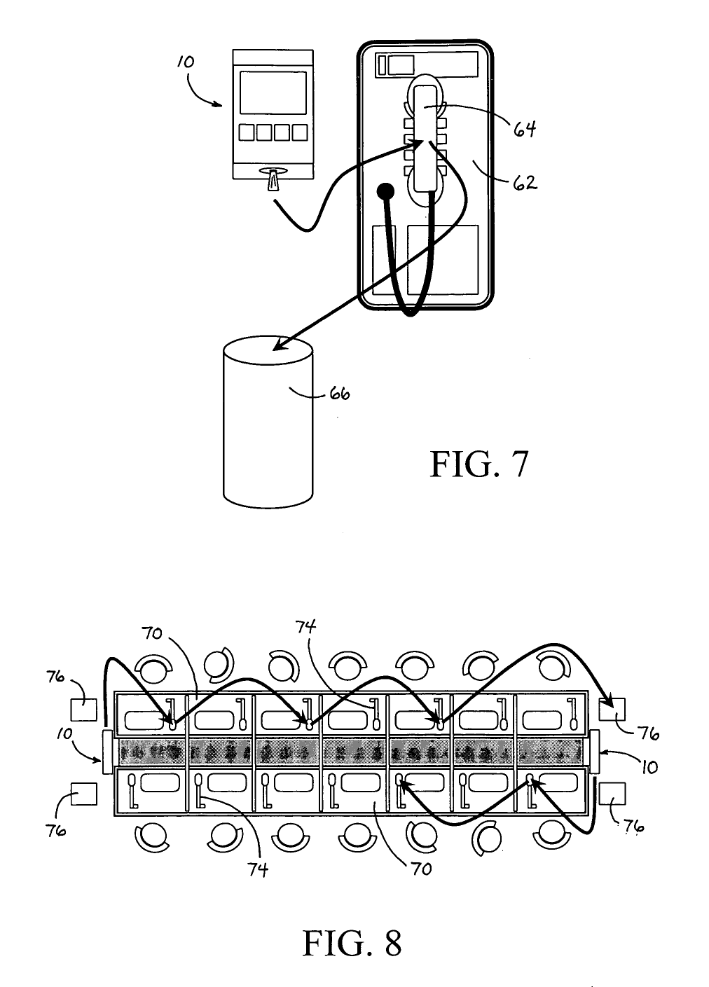

FIG. 7 shows a third preferred embodiment of the system in use with a public telephone 62. In this embodiment, the user pulls a towelette from the dispenser unit 10 positioned next to the public telephone 62. The user then uses the towelette to grasp the telephone handset 64. After completing usage of the telephone, the user disposes of the towelette in a refuse container 66 positioned next to the telephone.

FIG. 8 shows a fourth preferred embodiment of the system in use with a row of public casino slot machines 70. In this setting, the user pulls a towelette from the dispenser unit 10 positioned adjacent to the slot machines 70. The user then utilizes the towelette to grasp the slot machine handles 74, proceeding to the next slot machine and using the towelette to grasp its handle. After completion of gaming, the user disposes of the towelette in a refuse container 76 positioned adjacent to the row of slot machines.

In general, the systems and methods of the present invention provide a dermal defense barrier to microbial infection in any situation where an individual is forced to handle objects or touch surfaces previously handled or touched by others in the public. It is anticipated that further variations in both the structure of the dermal defense system and the methods of use of the system will be apparent to those skilled in the art from the present disclosure and the attached drawing figures. Such variations, while not explicitly described and defined herein, may be seen to fall within the spirit and scope of the present invention.

We claim:

1. A dermal defense system for use in conjunction with a public facility, the system comprising:
towelettes impregnated with an antimicrobial composition; and
a dispenser unit containing the towelettes positioned in close proximity to an object to be handled within the facility.

2. The dermal defense system of claim 1, wherein the public facility is a public restroom facility and the object to be handled is a door associated with an exit to the facility.

3. The dermal defense system of claim 1, wherein the public facility is a public dining facility and the object to be handled is at least one service utensil.

4. The dermal defense system of claim 1, wherein the public facility is a public telephone facility and the object to be handled is a telephone handset.

5. The dermal defense system of claim 1, wherein the public facility is a public casino slot machine facility and the object to be handled is at least one slot machine handle.

6. The dermal defense system of claim 1, 2, 3, 4, or 5, further comprising a refuse container for disposal of the towelettes.

7. The dermal defense system of claim 1, 2, 3, 4, or 5, wherein the antimicrobial composition is a mixture comprised of:
0.05-0.5% (w/v) Benzalkonium Chloride;
0.05-0.5% (w/v) Hymine (Benzethonium Chloride);
0.10-5.0% (w/v) Methylparaben;
0.01-0.5% (w/v) Propylparaben;
0.10-5.0% (w/v) Lanolin PEG 60;
0.10-5.0% (w/v) Aloe Barbadensis; and
further wherein the components are dissolved in purified water.

8. A method for dermal defense within a public facility, the method comprising the steps of:
providing towelettes impregnated with an antimicrobial composition;
positioning a dispenser unit containing the towelettes in close proximity to an object to be handled within the public facility; and
dispensing the towelettes impregnated with the antimicrobial composition from the dispenser unit to users of the public facility;
wherein the user grasps the towelette from the dispenser unit, positions the towelette as a barrier between the user's hand and the object to be handled, and disposes of the towelette after releasing the object to be handled.

9. The method of claim 8, wherein the public facility is a public restroom facility and the object to be handled is an exit door of the facility.

10. The method of claim 8, wherein the public facility is a public dining facility and the object to be handled is at least one service utensil.

11. The method of claim 8, wherein the public facility is a public telephone facility and the object to be handled is a handset of a telephone.

12. The method of claim 8, wherein the public facility is a casino slot machine facility and the object to be handled is at least one slot machine handle.

13. A dermal defense system comprising means for preventing the spread of dermally-transmitted microbes from an object to be handled by the public, the system comprising:
means for providing towelettes impregnated with an antimicrobial composition;
means for positioning a dispenser unit containing the towelettes next to the object to be handled by the public; and
means for dispensing the towelettes impregnated with the antimicrobial composition from the dispenser unit to individuals within the public;
wherein the individual grasps the towelette from the dispenser unit, positions the towelette as a barrier between the individual's hand and the object, and disposes of the towelette after releasing the object.
